(19) United States
(12) Patent Application Publication
(10) Pub. No.: US 2006/0094940 A1
(43) Pub. Date: May 4, 2006

(54) Method for selecting wavelengths for optical data acquisition

(75) Inventors: Philippe St-Jean, Lyon (FR); Frederic Lesage, Montreal (CA); Xavier Intes, Montreal (CA); Frederic Leblond, Montreal (CA)

Correspondence Address: BERESKIN AND PARR, 40 KING STREET WEST, BOX 401, TORONTO, ON, M5H 3Y2, CA

(73) Assignee: ART, ADVANCED RESEARCH TECHNOLOGIES, INC., Montreal (CA)

(21) Appl. No.: 11/230,646

(22) Filed: Sep. 21, 2005

Related U.S. Application Data
(60) Provisional application No. 60/611,294, filed on Sep. 21, 2004.

(51) Int. Cl. C12Q 1/00; G06F 19/00; A61B 5/00

(52) U.S. Cl. 600/315; 435/4; 702/19

(57) ABSTRACT

There is provided a method for optimizing wavelength selection for multiwavelength optical data acquisition of chromophores in a turbid medium. The optimization is based on the minimization of a criterion based on the variance matrix of chromophores estimate features. The method can advantageously be used to obtain physiological information from biological tissue.

### CROSS-REFERENCE TO RELATED APPLICATION

This patent application claims priority on U.S. provisional application No. 60/611,294 entitled “METHOD FOR SELECTING WAVELENGTHS FOR OPTICAL DATA ACQUISITION” and filed on Sep. 21, 2004.

### FIELD OF THE INVENTION

The present invention relates to the field of optical data acquisition, such as medical optical imaging, in which objects which diffuse light, such as some human body tissues, are probed using signals resulting from the injection of light into the object and detection of the diffusion of the light in the object at a number of positions. More particularly, the present invention relates to the choice of wavelengths for multiwavelength optical data acquisition, and in particular imaging, in order to provide enhanced information.

### BACKGROUND OF THE INVENTION

Optical medical imaging modalities such as Time-Domain (TD), Continuous Wave (CW) and Frequency-Domain (FD) show great promise as techniques for imaging breast tissue, as well as the brain and other body parts. In TD, the objective is to analyze the temporal point spread function (TPSF) of an injected pulse of light as it is diffused in the tissue. With CW, the attenuation of a continuous light source is measured. In FD the amplitude and phase of a frequency modulated signal is analyzed. The information extracted from these measurements is used in constructing medically useful images.

For example, one can extract time-gated attenuation information from the TPSF which provides high quality images albeit of lower resolution than other modalities such as X-ray imaging. Thus, it is unclear whether the spatial resolution provided by optical imaging is adequate for diagnosing breast cancer based on morphology.

Optical data, when processed adequately, can be used to extract absorption values from raw measurements. For example, the TD and FD signal can be used to decouple the light attenuation into absorption and scattering components. This extra information may be clinically useful. Moreover, one can obtain the tissue absorption spectrum by performing measurements at multiple wavelengths. Biological tissues comprise many natural near infrared chromophores. The contribution of each chromophore to the overall optical signal depends on the wavelength used, the absorption coefficient and the concentration of the chromophore. For example, the dominant near infrared chromophores contained in breast tissue are considered to be hemoglobin (Hb) in its oxygenated (HbO₂) and deoxygenated (HbR) forms, water and lipids. There are other interesting near infrared chromophores, such as glucose and cytochrome c oxidase, but their absorption contribution in the breast is considered negligible compared to the aforementioned chromophores. Spectroscopic analysis of the tissue absorption spectrum permits chromophore concentrations to be measured. Furthermore, combination of the chromophore concentrations can yield physiological information, as opposed to morphologic information, which could provide additional, medically useful information.

For example, total hemoglobin concentration in a tissue, [HbT], defined as [HbT]=[HbO₂]+[HbR], which can be obtained by optical measurements, is related to the local vascular density. Since cancer is commonly associated with an increase in vascularization (angiogenesis), a measurement of [HbT] could be medically useful. The fraction of hemoglobin that binds to oxygen is known as the oxygen saturation, S, and defined as S=[HbO₂]/[HbT]. Increased metabolic activity increases oxygen demands, which decreases the oxygen saturation. Since cancer is commonly associated with increased metabolic activity, a measurement of S could also be medically useful.

Historically, as the biomedical optics field evolved, the wavelengths were chosen for each chromophore individually by observing strong near infrared spectral features for the given chromophore and using the closest hardware-available wavelength. Many researchers also used the isobestic wavelength of oxy-Hb and deoxy-Hb, the wavelength where their absorption per concentration are equal, since this wavelength is insensitive to the oxygenation state of the hemoglobin and can be related to the [HbT].

However, the question both posed and addressed here is that for a given set of chromophores what are the optimal wavelengths to use in order to deduce information such as the concentration for each chromophore? It is interesting to note that the isobestic wavelength used by many researchers turns out not to be necessarily the best choice of wavelength. An approach based on the minimization of a condition number has been proposed in WO 2004/064626 A1 published on Aug. 5, 2004. However, the approach described in WO 2004/064626 A1 may lead to situations where the “optimal” set of wavelengths may be aberrant as will be shown below.

The problem is one of knowing which are the dominant chromophores to include in a tissue model and then choosing the “best” wavelengths to deduce their concentrations most accurately.

### SUMMARY OF THE INVENTION

In the present description by chromophore it is meant any molecule or complex of molecules capable of absorbing light and characterized by extinction coefficients that are function of wavelength. The term includes molecules that can absorb and emit light such as fluorophores. Furthermore, a molecule may behave as two or more separate chromophores if the spectral characteristics of the molecule are dependent on the physico-chemical environment of the molecule. For example the spectral characteristics of a molecule can change upon binding to another molecule.

It is an object of the invention to improve optical data acquisition for optically characterizing a turbid medium by choosing an efficient combination of wavelengths and combining information from the combination of wavelengths. For example optimal choice of wavelength may improve optical image quality in TD, FD or CW-based optical images.

It is an object of the present invention to provide an objective method for choosing the wavelengths for a multiwavelength TD, FD or CW-based optical imaging approach. For a given set of chromophores, the best selection of the wavelengths is performed for the set as a whole as opposed to choosing the best wavelength for each chromophore individually. Furthermore, hardware constraints can be taken into consideration in order to optimize the selection of wavelengths for a given device.

Thus in accordance with the present invention, there is provided a method for acquiring optical information from a turbid medium containing chromophores, in which the parameters of an optical system including at least a number N of said wavelengths, a value of each of said wavelengths, source power and detector aperture for each of said wavelengths, source/detector geometries, a choice of source and detector and noise characteristics are defined and the value for all of said parameters are fixed except for the value for each of the wavelengths. A chromophore estimate feature to minimize is then determined, a statistical distribution of errors for absorption coefficients of said chromophores is selected and an optimal set of N wavelengths is determined by minimizing a criterion based on the absorption coefficients as a function of wavelength, extinction coefficients of the chromophores and on a predetermined concentration value of the chromophores. The optimal set of N wavelengths is then used for obtaining the optical information.

The chromophore estimate feature can be selected from the group consisting of error on each chromophore, linear combination of errors on several chromophores, non-linear combinations of errors on one or several chromophores, correlation (cross-talk) between two chromophores, linear combination of correlations between chromophores, non-linear combination of correlations between chromophores and combination of errors and correlations between chromophores.

The selection of a statistical distribution of errors can be done by providing a predetermined distribution or based on empirical data.

The optical information obtained by the method of the present invention can be used to generate an optical image using an imaging optical system wherein the optical information is acquired with a modality selected from Time-Domain, Frequency-Domain and Continuous wave.

In one embodiment of the invention the optimized set of wavelengths is used to derive information from a biological tissue and in particular breast tissue or brain tissue. The chromophores can then be selected based on physiological characteristics of the tissue. For example, the chromophores are selected from oxy-hemoglobin, deoxy-hemoglobin, water, lipids and combination thereof.

### BRIEF DESCRIPTION OF THE DRAWINGS

Further features and advantages of the present invention will become apparent from the following detailed description, taken in combination with the appended drawings, in which:

FIG. 1 is a theoretical spectrum for two distinct chromophores;

FIG. 2 is a map of error for all combinations of wavelengths for our proposed criterion in which minima are located at the peak values as expected; and

FIG. 3 is a map of error for all combinations for the condition number criterion in which minima are found outside peak values at equal absorption values.

### DETAILED DESCRIPTION OF THE INVENTION

It is an object of the present invention to provide an objective method for choosing the wavelengths for deriving information about chromophores present in a biological tissue. Multiwavelength optimization is important for optical methods such as multiwavelength TD, FD or CW-based optical imaging approach. For a given set of chromophores, the best selection of the wavelengths is performed for the set as a whole as opposed to choosing the best wavelength for each chromophore individually. Moreover, it is also possible to investigate scenarios such as the influence on determining chromophore concentrations under certain assumptions about the concentration(s) of other chromophore(s) in the set.

In an embodiment of the invention, hardware constraints can also be taken into consideration in order to optimize the selection of wavelengths for a given device.

In the context of optical mammography, the chromophores of interest are oxyhemoglobin (HbO₂), deoxyhemoglobin (HbR), water and lipid. Absorption spectra for these chromophores in the range [λₘᵢₙ, λₘₐₓ] are used to construct a matrix A_{λ} of extinction coefficients that relates the vector of chromophore concentrations c to absorption coefficients μₐ through
μₐ=A_{λ}c. (1)

Errors Δμₐ on the estimation of μₐ propagate to the concentrations as μₐ+Δμₐ=A_{λ}(c+Δc). We begin by re-deriving the rationale for the condition number criterion. We write
Δμₐ=A_{λ}Δc, (2)
hence
Δc=A_{λ}⁻¹Δμₐ, (3)

where A_{λ}⁻¹ represents either the true inverse if it exists, or the pseudo inverse (A^{T}A)⁻¹A^{T}. In the latter case, the equality is understood as a minimum RMS solution. Taking the 2-norm on both sides gives

$$\begin{matrix}‖ Δ c ‖ = ‖ {A}_{λ}^{- 1} {Δμ}_{a} ‖ , & ( 4 ) \\ ‖ Δ c ‖ \leq ‖ {A}_{λ}^{- 1} ‖ ‖ {Δμ}_{a} ‖ . & ( 5 )\end{matrix}$$

Similarly,
∥μₐ∥≦∥A_{λ}∥∥c∥. (6)

Putting these last two equations together yields

$$\frac{‖ Δ c ‖}{‖ c ‖} \leq ‖ {A}_{λ}^{- 1} ‖ ‖ {A}_{λ} ‖ \frac{‖ {Δμ}_{a} ‖}{‖ {μ}_{a} ‖} . \qquad ( 7 )$$

The form ∥A_{λ}⁻¹∥∥A_{λ}∥ defines the condition number of A_{λ}. As the 2-norm of a matrix is given by its largest singular value, the condition number is readily obtained by computing the ratio of the largest singular value of A_{λ} to its smallest singular value, as the largest singular value of the inverse is the reciprocal of the smallest.

It can then be argued that by making the condition number as small as possible we get the lowest boundary over the error Δc that we are trying to minimize. However, there are two possible inconvenients in using this criterion. First, expression (7) only provides a boundary on the error; if this boundary is not tight enough, there is no guarantee that the truly optimal choice for the wavelengths is indeed the one that minimizes the condition number. Second, it attempts to minimize the ratio of the error norm over the concentration norm

$$\frac{‖ Δ c ‖}{‖ c ‖} , \qquad ( 8 )$$

However, it has been discovered that it is preferable to minimize the norm of the ratios,

$$‖ \frac{Δ c}{c} ‖ \qquad ( 9 )$$

Example 1 below provides a simple example where the condition number fails to provide the most desirable optimization.

In an aspect of the present invention a different approach is proposed that advantageously avoids the difficulties and limitations posed by the condition number method.

The variance matrix associated with the μₐ measurements for all operative wavelengths is given by the expression

$${\sum }_{AB} = 〈 {( {μ}_{a} - {\overline{μ}}_{a} )}_{A} {( {μ}_{a} - {\overline{μ}}_{a} )}_{B}^{T} 〉 , \qquad ( 10 )$$

where < > is the expectation value operator, A, B=1, . . . , N_{λ}, N_{λ} Is the number of wavelengths (number of μₐ measurements) and

$${μ}_{a} - {\overline{μ}}_{a} = ( \begin{matrix}{μ}_{a} ( {λ}_{1} ) - {\overline{μ}}_{a} ( {λ}_{1} ) \\ ⋮ \\ {μ}_{a} ( {λ}_{{N}_{λ}} ) - {\overline{μ}}_{a} ( {λ}_{{N}_{λ}} )\end{matrix} ) = ( \begin{matrix}{Δμ}_{a} ( {λ}_{1} ) \\ ⋮ \\ {Δμ}_{a} ( {λ}_{{N}_{λ}} )\end{matrix} ) . \qquad ( 11 )$$

Then the expression (10) can be written like

$${\sum }_{AB} = ( \begin{matrix}{〈 {Δμ}_{a} ( {λ}_{1} ) 〉}^{2} & … & 〈 {Δμ}_{a} ( {λ}_{1} ) \cdot {Δμ}_{a} ( {λ}_{{N}_{λ}} ) 〉 \\ ⋮ & ⋰ & ⋮ \\ 〈 {Δμ}_{a} ( {λ}_{1} ) \cdot {Δμ}_{a} ( {λ}_{{N}_{λ}} ) 〉 & ⋯ & {〈 {Δμ}_{a} ( {λ}_{{N}_{λ}} ) 〉}^{2}\end{matrix} ) , \qquad ( 12 )$$

which is a symmetric and positive-definite matrix. The diagonal elements of Σ are the variances associated with the randomly distributed variables Δμₐ(λ_{A}) while the off-diagonal elements are the covariances (proportional to the correlation factors) between the random variables at different wavelengths. For simplicity we use the notation,

$$\begin{matrix}{〈 {Δμ}_{a} ( {λ}_{A} ) 〉}^{2} = {σ}_{A}^{2} & ( 13 ) \\ 〈 {Δμ}_{a} ( {λ}_{A} ) \cdot {Δμ}_{a} ( {λ}_{B} ) 〉 = {σ}_{{AB}^{′}} & ( 14 )\end{matrix}$$

so the covariance matrix takes the form

$${\sum }_{AB} = ( \begin{matrix}{σ}_{1}^{2} & ⋯ & {σ}_{1 {N}_{λ}} \\ ⋮ & ⋰ & ⋮ \\ {σ}_{1 {N}_{λ}} & ⋯ & {σ}_{{N}_{λ}}^{2}\end{matrix} ) . \qquad ( 15 )$$

The particular statistics such as normal, Poisson, Gaussian, binomial and the like chosen for the random variables Δμₐ(λ_{A}) will be reflected in the values used for the elements of the variance matrix Σ. For example, in the case of a multivariate distribution with zero mean, zero covariance and equal variances (σ²) we have

$${\sum }_{AB} = ( \begin{matrix}{σ}^{2} & 0 & 0 \\ 0 & ⋰ & 0 \\ 0 & 0 & {σ}^{2}\end{matrix} ) . \qquad ( 16 )$$

The expectation (mean) value of the percentage error on the chromophores is

$$〈 {‖ \frac{Δ c}{c} ‖}_{2} 〉 = 〈 {‖ {C}^{- 1} Δ c ‖}_{2} 〉 , \qquad ( 17 )$$

where Cᵢᵢ=cᵢ, i.e. C is a diagonal matrix filled with the elements of the concentration vector c. We can rewrite this as

$$〈 {‖ \frac{Δ c}{c} ‖}_{2} 〉 = 〈 {‖ {C}^{- 1} {A}_{λ}^{- 1} {Δμ}_{a} ‖}_{2} 〉 , \qquad ( 18 )$$

Thus the corresponding variance matrix of the percentage error is expressed as

$${( \frac{Δ c}{c} )}_{Ai} = {C}^{- 1} {A}_{λ}^{- 1} {Σ ( {A}_{λ}^{- 1} )}^{T} {C}^{- 1} , \qquad ( 19 )$$

where i=1, . . . , N_{C} with N_{C} representing the number of chromophores assumed to contribute to the absorption coefficient μₐ. Thus the criterion to minimize is the variance matrix of the chromophore estimate feature (here the error on the concentration).

In one example, the expected value of the 2-norm of a multivariate normal is the trace of its variance matrix, hence a criterion to minimize is

$$Arg {min}_{λ} 〈 {‖ \frac{Δ c}{c} ‖}_{2} 〉 = Arg {min}_{λ} ( tr ( {C}^{- 1} {A}_{λ}^{- 1} {Σ ( {A}_{λ}^{- 1} )}^{T} {C}^{- 1} ) ) . \qquad ( 20 )$$

Note that if Σ=σ²l, the trace can be written as

$${σ}^{2} \cdot tr ( {C}^{- 2} {VS}^{- 2} {V}^{T} ) \qquad ( 21 )$$

where V and S are the usual SVD matrices of A_{λ}.

This matrix is by definition symmetric and positive-definite. The variance matrix indicates how measurement errors and uncertainties associated with μₐ measurements will propagate in the calculation of the associated chromophore concentrations.

The variance matrix is wavelength-dependent. This dependence is brought by the spectral behavior of the extinction coefficients and the particular statistics associated with the μₐ measurements.

The diagonal elements of the variance matrix are associated with the standard deviations (variances) related to each chromophore. Taken individually these elements represent a measure for how much the μₐ errors are propagating into each chromophore.

The off-diagonal elements represent the covariance between the different chromophores. The amplitude of these elements is associated with a measure of the correlation between the chromophores. It will be appreciated that this correlation can be used to quantify the cross-talk between the physiological parameters associated with these chromophores.

Different wavelength sets can be selected in order to minimize specific elements (or combinations thereof of the variance matrix. Thus by appropriately choosing the wavelengths one can effectively minimize particular features of the μₐ—chromophores inverse problem. Here are some examples of the particular chromophore estimate features that can be minimized when a wavelength set is selected:

error on each chromophore, linear combination of errors on several chromophores, non-linear combinations of errors on one or several chromophores, correlation (cross-talk) between two chromophores, linear combination of correlations between chromophores, non-linear combination of correlations between chromophores, combination of errors and correlations between chrombphores.

While the above embodiment of the invention is described using a multivariate statistics with vanishing covariances, it will be appreciated that other statistical error distribution can be selected (such as normal, Poisson and the like). The selection may either be based in theoretical considerations or be based from empirical data obtained from the absorption measurements of chromophores.

It is important to notice that the concentrations c explicitly appears in the proposed minimization, as opposed to the one based upon the condition number criterion.

If even after properly taking into account typical concentration sone of the chromophore happens to be significantly less absorbant than the others, it will completely dominate the minimization. This is due to the fact that this chromophore then becomes the one for which bounding the error proves to be the most difficult. For instance, this is usually the case with lipids in the context of optical mammography. However, lipid concentration is believed (to this day) to be less important from a diagnostic point of view than the other chromophores. Hence a somewhat larger error on its concentration is acceptable.

The criterion used in the present invention allows to take this into account by weighting the percentage error in equation 9 for each individual chromophore. Moreover, the matrix from which the trace is taken holds on its diagonal variance for each individual chromophore. Thus not only can one minimize a weighted sum of the diagonal, but non-linear function of those individual variances can also be used in the minimization, as might be the case when looking for minimal error over oxygen saturation, for instance.

In the case of an over-constrained problem, i.e. when there are more wavelengths than chromophores, the criterion of the present invention allows for the selection of the same wavelength multiple times. This is not necessarily a shortcoming of the criterion. For instance, when there is one more wavelength than there are chromophores, using the same wavelength twice amounts to increase the spectra by a factor of \sqrt{2} at this wavelength and working with a square matrix, as far as the minimization is concerned. Practically, this means that a reconditioning of the inverse matrix (now square, of the dimension given by the number of chromophores) could lead to better result, assuming that SNR is also improved at that wavelength. This can possibly be achieved by longer acquisition time at that wavelength.

The concentration of the chromophores for use in the minimization described above may be estimated or it may be experimentally determined. It will be appreciated that the term chromophores comprises any molecule capable of absorbing light, including fluorophores.

It will be appreciated that while the above described criterion is based on the concentration and therefore μₐ of the chromophores, the optimized set of wavelength may also be used to derive optical properties other than absorption. For example, optimization of wavelength selection based on absorption may be useful to improve optical measurements to obtain scatter values as for example when the scatter value is derived in part by using expressions exhibiting a dependence on absorption coefficient. In general the optical information acquired by the method of the present invention can be used for various purposes such as optical imaging and the acquisition of functional physiological information (concentration, saturation levels etc.).

Many of the applications of the method described above may require the use near infrared radiation (NIR) but the method is not limited to this region of the spectrum and can be applied to any optical wavelength. Furthermore the modality of optical information acquisition may comprise Continuous wave (CW), Time-domain (TD) and Frequency-Domain (FD) or any combination thereof.

In another aspect of the invention, the method of the present invention can be applied to select a set of optimal wavelengths for obtaining pharmacological/physiological information derived from the state of a particular molecule. For example, the binding of a fluorophore to a target molecule may give rise to changes in the spectral characteristics of the fluorophore and it may be desirable to optimize the wavelengths used to characterize the binding. Furthermore the binding may also be studied in conjunction with other physiological properties (levels of hemoglobin for example) that may require further wavelength optimization.

### EXAMPLE 1

Optimisation Based Upon the Condition Number:

| | | | | |
|---|---|---|---|---|
| wavelengths (nm): | 735 | 760 | 795 | 835 |

expected %-error of the concentrations:

| | |
|---|---|
| HbR (deoxy-haemoglobin): | 2.9987 |
| HbO2 (oxy-haemoglobin): | 5.9960 |
| Water: | 25.6071 |
| Lipid: | 26.1913 |

condition number for this set: 36.3741

Optimisation Based Upon the Proposed Criterion:

| | | | | |
|---|---|---|---|---|
| wavelengths (nm): | 690 | 735 | 760 | 805 |

expected %-error of the concentrations:

| | |
|---|---|
| HbR (deoxy-haemoglobin): | 1.2041 |
| HbO2 (oxy-haemoglobin): | 4.7867 |
| Water: | 19.1384 |
| Lipid: | 24.7224 |

condition number for this set: 42.7687

### EXAMPLE 2

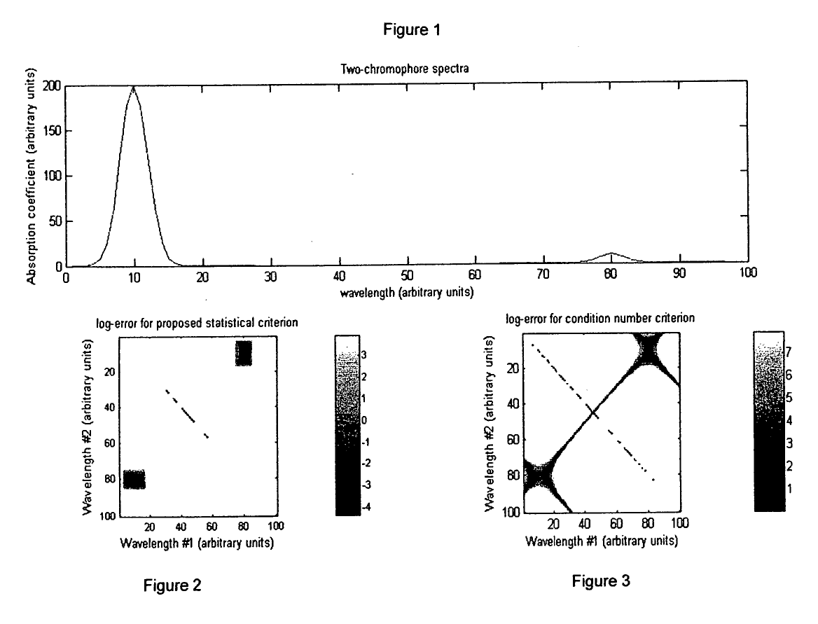

In another example let us assume that there are only two chromophores and two wavelengths (see FIG. 1). We also assume that the absorption spectra for those chromophores come in the form of delta-like function, i.e. each chromophore only absorbs close to one specific wavelength, and those two wavelengths are well separated. Finally, we assume that one of the two chromophores is overall much less absorbent than the other, each taken at their respective absorption wavelength.

Obviously, the optimal choice of wavelength in this case should be the peak values of each spectrum. However, the condition number criterion will not give that result. Take a₁ and a₂ to be the peak values of the spectra of the two chromophores, with α₁ α_{2.} In this case, the matrix A has α₁ and α₂ on the diagonal, and zero everywhere else. The condition number of this matrix is trivially found to be equal to α₂/α₁, a large number. Yet if we were to choose an off-peak value for the second chromophore, off enough that α₂ becomes equal to α₁, then the condition number becomes equal to one, which is minimal. Coming back to expressions (8,9), one readily sees that this awkward choice is relevant of the minimization of the first type of error; it does not minimize for the second, correct one.

### EXAMPLE 3

In the text above, we proposed a criterion for wavelength optimization that differs from the standard condition number criterion, avoiding certain problems encountered when using the latter. We present here comparison between the two methods, and conclude by proposing new set of optimal wavelengths.

We present four different optimization schemes:

One with standard concentrations of the four chromophores, namely deoxyhemoglobin (HbR) and oxyhemoglobin (HbO₂) at 1.0 micro-Mole water at 18% and lipid at 70%. Since absorption of lipids is much smaller than the three other chromophores at these typical concentrations (using the olive oil spectrum), the optimization is completely dominated by the former while it also is the least significant diagnostically.

We propose a second scheme where only the error over HbR, (HbO₂) and water is minimized, which leads to a second set of wavelengths.

A third set is obtained in the case where assign weights of [1,1,0.5,0.1] to HbR, (HbO₂), water and lipids respectively. These are ad-hoc parameters reflecting diagnostic importance of the chromophores. This third set also allows to overcome a possible dominance of the water over the two types of blood when the lipid is not taken into account.

Finally, assuming that the lipid background can be estimated and its contribution subtracted from the estimated μₐ at every wavelength, an over-constrained minimization is obtained with the three remaining chromophores still using four wavelengths. This is similar to what was done in Corlu [A. Corlu et al., Opt. Lett. Vol. 28, No. 23, 2339 (2003)].

A specific example of how these wavelengths can be optimized is presented below for a variable wavelength laser technology (MAITAI laser). In this example, the wavelength range is constrained to [750,850]nm due to properties of the laser. A first set of four wavelengths that was used in clinical settings is (760,780,830,850)nm. This set is compared to the different optimization schemes. Table (1) shows the results of the analysis in that case as well as for a different set of wavelengths. The last column, entitled “RMS %-error”, gives the standard deviation on the chromophore concentrations corresponding to a 5.0E-5 mm⁻¹ standard deviation on μₐ estimation, regardless of the wavelength. This translates on average to a 1% standard deviation for total absorption in the range [750,850]nm, using the same standard concentrations as above.

TABLE 1
Results for the different criterion.

| | | wavelengths | Condition | |
|---|---|---|---|---|
| Scheme | criterion | independently (nm) | number | RMS %-error |
| A | CN | [760, 780, 820, 850] | 38.15 | [0.0304, 0.0604, |
| | | | | 0.249, 0.279] |
| A | % RMS | [760, 780, 820, 850] | 38.15 | [0.0304, 0.0604, |
| | | | | 0.249, 0.279] |
| B | % RMS | [750, 805, 820, 850] | 87.08 | [0.0591, 0.0491, |
| | | | | 0.206, 0.698] |
| C | % RMS | [750, 795, 820, 850] | 52.76 | [0.0367, 0.0521, |
| | | | | 0.224, 0.413] |
| D | % RMS | [750, 805, 805, 850] | 23.61 | [0.0250, 0.0410, |
| | | | | 0.188, —] |
| Current | — | [760, 780, 830, 850] | 77.70 | [0.0279, 0.1400, |
| | | | | 0.532, 0.316] |

Note in this case that scheme A yields the same results with both criteria. Scheme B optimizes for water (and some gain on (HbO₂)) at the cost of additional error over HbR, which is unwished for. Moreover, the condition number goes high. Even though we believe the % RMS criterion to be better suited to error minimization, it is dependent on a noise model with zero-mean. A systematic estimation error over the μₐ can appear, which is somewhat controlled by the condition number. Hence it is preferable that it remains low. Scheme C presents slightly better control over (HbO2), and water than scheme A, yet the condition number goes higher. Scheme D presents an overall improvement, but is dependent on proper background estimation of lipid concentration. Note that the 805 nm wavelength is repeated twice, this is not a mistake; see above for an explanation.

When we use laser diodes, we can enlarge the range of wavelengths accessible. Here the detection system might limit the range. We performed the same analysis by selecting wavelengths in the range [680,850]nm. Table (2) shows the results in that case. The data is presented in the same way as in Table (1).

TABLE 2
Results for the enlarge wavelength choices.

| | | | Condition | |
|---|---|---|---|---|
| Scheme | Criterion | Wavelengths | number | RMS %-error |
| A | CN | [760, 780, | 38.15 | [0.0106, 0.0554, 0.212, |
| | | 820, 850] | | 0.263] |
| A | RMS | [680, 740, | 51.78 | [0.0147, 0.0481, 0.165, |
| | | 760, 800] | | 0.779] |
| B | RMS | [700, 740, | 101.99 | [0.0591, 0.0491, 0.206, |
| | | 820, 850] | | 0.698] |
| C | RMS | [680, 740, | 52.42 | [0.0096, 0.0439, 0.189, |
| | | 760, 820] | | 0.290] |
| D | RMS | [680, 740, | 20.19 | [0.0095, 0.0411, |
| | | 740, 810] | | 0.127, —] |

In this case scheme A does not give the same result using the condition number VS the %-RMS criterion; one can see however that every chromophores show better result with the latter. This is a good example of the rationale for the new criterion, as discussed previously Here again, scheme B mostly tries to improve on water. Scheme C gives slightly better result than all the preceding ones, except for a small increase of the error for the lipids, which is acceptable. Still scheme D is the best but relies on background estimate of the lipid content.

While the invention has been described in connection with specific embodiments thereof, it will be understood that it is capable of further modifications and this application is intended to cover any variations, uses, or adaptations of the invention following, in general, the principles of the invention and including such departures from the present disclosures as come within known or customary practice within the art to which the invention pertains and as may be applied to the essential features herein before set forth, and as follows in the scope of the appended claims.

1. A method for acquiring optical information from a turbid medium containing chromophores, the method comprising the steps of:
a) defining parameters of an optical system including at least a number N of said wavelengths, a value of each of said wavelengths, source power and detector aperture for each of said wavelengths, source/detector geometries, a choice of source and detector and noise characteristics;
b) fixing a value for all of said parameters except for said value for each of the wavelengths;
c) determining a chromophore estimate feature to minimize;
d) selecting a statistical distribution of errors for absorption coefficients of said chromophores;
e) determining an optimal set of N wavelengths by minimizing a criterion, wherein said criterion is based on said absorption coefficients as a function of wavelength, extinction coefficients of said chromophores and on a predetermined concentration value of said chromophores;
f) using said optimal set of N wavelengths for obtaining said optical information.

2. The method as claimed in claim 1 wherein said chromophore estimate feature is selected from the group consisting of error on each chromophore, linear combination of errors on several chromophores, non-linear combinations of errors on one or several chromophores, correlation (cross-talk) between two chromophores, linear combination of correlations between chromophores, non-linear combination of correlations between chromophores and combination of errors and correlations between chromophores.

3. The method as claimed in claim 2 wherein said step of selecting a statistical distribution of errors comprises providing a predetermined distribution.

4. The method as claimed in claim 2 wherein said selection of statistical distribution of errors is determined based on empirical data.

5. The method as claimed in claim 3 or 4 wherein said statistical distribution of errors is a mutlivariate statistic.

6. The method as claimed in claim 5 wherein said multivariate statistic is selected from normal distribution, Poisson distribution, Gaussian distribution and binomial distribution.

7. The method as claimed in claim 1 wherein said optical information is used to generate an optical image and wherein said optical system is an imaging optical system.

8. The method as claimed in claim 1 wherein said optimal set of N wavelengths is used for determining concentration of said chromophores.

9. The method of claim 5 wherein said statistical distribution of errors is a multivariate normal of zero mean and variance and wherein said criterion is (tr(C⁻¹A⁻¹_{λ}Σ(A⁻¹_{λ})^{T}C⁻¹)).

10. The method as claimed in claim 1 wherein said turbid medium is a biological tissue.

11. The method as claimed in claim 10 wherein said biological tissue is selected from breast tissue and brain tissue.

12. The method as claimed in claim 11 wherein said chromophores are selected based on physiological characteristics of said tissue.

13. The method as claimed in claim 12 wherein said chromophores are selected from oxy-hemoglobin, deoxy-hemoglobin, water, lipids and combination thereof.

14. The method as claimed in claim 1 wherein said chromophores comprise at least one fluorophore.

15. The method as claimed in claim 14 wherein said fluorophore exhibits different spectral characteristics that differ in its free and bound state.

16. The method as claimed in claim 15 said optical information is used to generate pharmacological data.

17. The method as claimed in claim 1 wherein said optical information is acquired with a modality selected from Time-Domain, Frequency-Domain and Continuous wave.
